SEQUENCE LISTING (11 sequences)

SEQ ID NO: 1 - DNA, 34 bp, Artificial Sequence
  Description of Artificial Sequence Synthetic puro-loxP oligonucleotide
ataacttcgtatagcatacattatacgaagttat

SEQ ID NO: 2 - DNA, 2097 bp, Artificial Sequence
  Description of Artificial Sequence Synthetic transferrin-open reading frame polynucleotide
atgaggctcgccgtgggagccctgctggtctgcgccgtcctggggctgtgtctggctgtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacctggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctggacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtcccccccaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagaccttaa

SEQ ID NO: 3 - DNA, 3279 bp, Artificial Sequence
  Description of Artificial Sequence Synthetic Chimeric receptor ErbB2V>E/IGF-IR polynucleotide
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggaaggcattctgctggtcgtggtcttgggggtggtctttgggatcctcatcaagcgacggcagcagaagattgctagcagaaagagaaataacagcaggctggggaatggagtgctgtatgcctctgtgaacccggagtacttcagcgctgctgatgtgtacgttcctgatgagtgggaggtggctcgggagaagatcaccatgagccgggaacttgggcaggggtcgtttgggatggtctatgaaggagttgccaagggtgtggtgaaagatgaacctgaaaccagagtggccattaaaacagtgaacgaggccgcaagcatgcgtgagaggattgagtttctcaacgaagcttctgtgatgaaggagttcaattgtcaccatgtggtgcgattgctgggtgtggtgtcccaaggccagccaacactggtcatcatggaactgatgacacggggcgatctcaaaagttatctccggtctctgaggccagaaatggagaataatccagtcctagcacctccaagcctgagcaagatgattcagatggccggagagattgcagacggcatggcatacctcaacgccaataagttcgtccacagagaccttgctgcccggaattgcatggtagccgaagatttcacagtcaaaatcggagattttggtatgacgcgagatatctatgagacagactattaccggaaaggagggaaagggctgctgcccgtgcgctggatgtctcctgagtccctcaaggatggagtcttcaccacttactcggacgtctggtccttcggggtcgtcctctgggagatcgccacactggccgagcagccctaccagggcttgtccaacgagcaagtccttcgcttcgtcatggagggcggccttctggacaagccagacaactgtcctgacatgctgtttgaactgatgcgcatgtgctggcagtataaccccaagatgaggccttccttcctggagatcatcagcagcatcaaagaggagatggagcctggcttccgggaggtctccttctactacagcgaggagaacaagctgcccgagccggaggagctggacctggagccagagaacatggagagcgtccccctggacccctcggcctcctcgtcctccctgccactgcccgacagacactcaggacacaaggccgagaacggccccggccctggggtgctggtcctccgcgccagcttcgacgagagacagccttacgcccacatgaacgggggccgcaagaacgagcgggccttgccgctgccccagtcttcgacctgctga

SEQ ID NO: 4 - DNA, 65 bp, Artificial Sequence
  Description of Artificial Sequence Synthetic primer
ttttttcctgcaggtattatcaccggtgttttggcgcgccaggtggcacttttcggggaaatgtg

SEQ ID NO: 5 - DNA, 22 bp, Artificial Sequence
  Description of Artificial Sequence Synthetic primer
agctgcaagaactcttcctcac

SEQ ID NO: 6 - DNA, 15 bp, Artificial Sequence
  Description of Artificial Sequence Synthetic primer
tcaggcaccgggctt

SEQ ID NO: 7 - DNA, 21 bp, Artificial Sequence
  Description of Artificial Sequence Synthetic primer
cccgccttcctggagacctcc

SEQ ID NO: 8 - DNA, 18 bp, Artificial Sequence
  Description of Artificial Sequence Synthetic primer
cgccccgcaacctcccct

SEQ ID NO: 9 - protein, 1255 aa, Homo sapiens [sequence omitted]

SEQ ID NO: 10 - protein, 1367 aa, Homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 11 - DNA, 61 bp, Artificial Sequence
  Description of Artificial Sequence Synthetic primer
ataatacctgcaggaaaaggccggccaaaggatccctgtggaatgtgtgtcagttagggtg

(19) United States
(12) Patent Application Publication
(10) Pub. No.: US 2010/0178672 A1
(43) Pub. Date: Jul. 15, 2010

(54) CHO CELL

(76) Inventors: Robert DeLange, Penzberg (DE); Erhard Kopetzki, Penzberg (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 12/664,694

(22) PCT Filed: Jun. 25, 2008
(86) PCT No.: PCT/EP08/05134

§ 371 Date: Dec. 15, 2009

(30) Foreign Application Priority Data
Jun. 29, 2007 (EP) 07012773.3

(51) Int. Cl. C12P 21/06; C12N 5/07; C12N 15/00

(52) U.S. Cl. 435/69.1; 435/358; 435/361; 435/455

(57) ABSTRACT

The current invention comprises a CHO cell which is expressing a constitutively active mitogenic receptor, a method of obtaining such a CHO cell, and a method for the expression of a heterologous polypeptide using a CHO cell according to the invention.

The current invention provides a CHO cell possessing a constitutively active mitogenic receptor. This cell is useful for protein expression. Therefore the current invention is in the fields of mammalian cell engineering and protein expression.

### BACKGROUND OF THE INVENTION

Expression systems for the production of recombinant polypeptides are well-known in the state of the art and are described by, e.g., Marino, M. H., Biopharm. 2 (1989) 18-33; Goeddel, D. V., et al., Methods Enzymol. 185 (1990) 3-7; Wurm, F., and Bernard, A., Curr. Opin. Biotechnol. 10 (1999) 156-159. Such an expression system comprises a host cell and a suitable expression plasmid.

The essential elements of an expression plasmid are a prokaryotic plasmid propagation unit for example for E. coli comprising an origin of replication and a selection marker, an eukaryotic selection marker, and one or more expression cassettes for the expression of the structural gene(s) of interest each comprising a promoter, a structural gene, and a transcription terminator including a polyadenylation signal. For transient expression in mammalian cells a mammalian origin of replication, such as the SV40 Ori or OriP, can be included. As promoter a constitutive or inducible promoter can be selected. For optimized transcription a Kozak sequence may be included in the 5′ untranslated region. For mRNA processing, in particular mRNA splicing and transcription termination, mRNA splicing signals, depending on the organization of the structural gene (exon/intron organization), may be included as well as a polyadenylation signal.

Polypeptides for use in pharmaceutical applications are preferably produced in mammalian cells such as CHO cells, NS0 cells, Sp2/0 cells, COS cells, HEK cells, BHK cells, PER.C6® cells, or the like. For the fermentation of the host cell and thus for the expression of the polypeptide of interest a cultivation medium is used.

Today CHO cells are widely used for the expression of pharmaceutical polypeptides, either at small scale in the laboratory or at large scale in production processes. Due to their wide distribution and use the characteristic properties and the genetic background of CHO cells is well known. Therefore, CHO cells are approved by regulatory authorities for the production of therapeutic proteins for application to human beings.

But there are still a lot of provisos with respect to the culture medium. By the use of animal-derived components the potential risk of contamination with substances hazardous to humans, such as viruses or prion proteins, is at hand. Besides the high costs and downstream processing problems another problem of animal-derived components is due to batch-to-batch variations as natural product making it difficult to obtain a constant product quantity and quality.

To overcome these provisos production cell lines are needed which do require fewer animal-derived components for their cultivation.

A super CHO cell line capable of autocrine growth under fully defined protein-free conditions has been reported by Pak, et al., (Pak, S. C. O., et al., Cytotechnology 22 (1996) 139-146). This is a CHO-K1 (ATCC CCL 61) cell line expressing transferrin and IGF-I. Morris, A. E., et al. (US 2005/0170462) report a method for the recombinant protein production in cell culture by modulation of the IGF-I signaling pathway. Belaus et al. transfected an IL-3 dependent murine BaF/3 cell with a chimeric ErbB2^{V→E}/IGF-I receptor (Belaus, A., et al., J. Steroid. Biochem. Mol. Biol. 85 (2003) 105-115).

### SUMMARY OF THE INVENTION

The current invention provides a CHO cell with a constitutively active mitogenic receptor, a method for obtaining such a CHO cell, and a method for the recombinant production of a heterologous polypeptide in such a CHO cell.

One aspect of the current invention is a CHO cell, which is expressing a constitutively active mitogenic receptor. In one embodiment the CHO cell is a CHO-K1 cell. In another embodiment the constitutively active mitogenic receptor is a chimeric ErbB2^{V→E}/IGF-I receptor.

A further aspect of the current invention is the CHO cell line DSM ACC2851.

Another aspect of the current invention is a method for obtaining a CHO cell with a constitutively active mitogenic receptor, wherein the method comprises the following steps:
a) transfecting a CHO cell with a first nucleic acid comprising
   i) an expression cassette encoding a selectable marker flanked by two loxP-sites,
   ii) an expression cassette for the expression of a constitutively active mitogenic receptor,
b) selecting a CHO cell transfected with the first nucleic acid,
c) transfecting the CHO cell selected in step b) with a second nucleic acid comprising a CRE recombinase expression cassette,
d) selecting a CHO cell transfected with the second nucleic acid as a CHO cell with a constitutively active mitogenic receptor.

In one embodiment the transfected CHO cell of step b) has a constitutively active mitogenic receptor. In another embodiment the transfected CHO cell is selected in step d) for the absence of the selectable marker comprised in the first nucleic acid. In still another embodiment the transfected CHO cell selected in step d) is not growing in the presence of a selection agent according to the selectable marker of the first nucleic acid. In a further embodiment comprises the first nucleic acid an additional expression cassette for the expression of transferrin.

A further aspect of the current invention is a method for the recombinant production of a heterologous polypeptide in a CHO cell according to the invention which is expressing a constitutively active mitogenic receptor. In one embodiment the nucleic acid with which the CHO cell is transfected comprises an expression cassette encoding a heterologous polypeptide. In one embodiment the heterologous polypeptide is a human polypeptide. In another embodiment the heterologous polypeptide is selected from the polypeptides comprising immunoglobulins, immunoglobulin heavy chains, immunoglobulin light chains, immunoglobulin fragments, or immunoglobulin conjugates.

### DETAILED DESCRIPTION OF THE INVENTION

The current invention comprises a CHO cell, which is expressing a constitutively active mitogenic receptor.

Methods and techniques known to a person skilled in the art, which are useful for carrying out the current invention, are described e.g. in Ausubel, F. M., ed., Current Protocols in Molecular Biology, Volumes I to III (1997), Wiley and Sons; Sambrook et al., Molecular Cloning: A Laboratory Manual, Second Edition, Cold Spring Harbor Laboratory Press, Cold Spring Harbor, N.Y. (1989).

The term “nucleic acid” as used herein, denotes a polymer consisting of individual nucleotides, i.e. a polynucleotide. It refers to a naturally occurring, or partially or fully non-naturally occurring nucleic acid, which is e.g. encoding a polypeptide that can be produced recombinantly. The nucleic acid can be build up of DNA-fragments which are either isolated or synthesized by chemical means. The nucleic acid can be integrated into another nucleic acid, e.g. in an expression plasmid or the genome/chromosome of a host cell. Plasmid includes shuttle and expression vectors. Typically, the plasmid will also comprise a prokaryotic propagation unit comprising an origin of replication (e.g. the ColE1 origin of replication) and a selectable marker (e.g. ampicillin or tetracycline resistance gene) for replication and selection, respectively, of the vector in bacteria. A nucleic acid is likewise characterized by its sequence consisting of individual nucleotides or by the amino acid sequence encoded by the nucleic acid molecule.

An “expression cassette” refers to a nucleic acid that contains the elements necessary for expression and secretion of at least the contained structural gene in a host cell.

A “gene” denotes a nucleic acid which is a segment e.g. on a chromosome or on a plasmid which can effect the expression of a peptide, polypeptide, or protein. Beside the coding region, i.e. the structural gene, a gene comprises other functional elements e.g. a signal sequence, promoter(s), introns, and/or terminators.

A “structural gene” denotes the region of a gene without a signal sequence, i.e. the coding region.

The term “selectable marker” denotes a nucleic acid that allows cells carrying this nucleic acid to be specifically selected for or against, in the presence of a corresponding “selection agent”. A useful positive selectable marker is e.g. an antibiotic resistance gene. The selectable marker allows a cell which is transformed therewith to be selected for in the presence of the corresponding selection agent; a non-transformed cell is not capable to grow or survive under these selective culture conditions. Selectable markers can be positive, negative or bifunctional. Positive selectable markers allow the selection of cells carrying the marker, whereas negative selectable markers allow cells carrying the marker to be selectively eliminated. Typically, a selectable marker will confer resistance to a drug or compensate for a metabolic or catabolic defect in the cell. Selectable markers useful with eukaryotic cells include, e.g., the genes for aminoglycoside phosphotransferase (APH), such as the hygromycin phosphotransferase (hyg), neomycin and G418 APH, dihydrofolate reductase (DHFR), thymidine kinase (tk), glutamine synthetase (GS), asparagine synthetase, tryptophan synthetase (selection agent indole), histidinol dehydrogenase (selection agent histidinol D), and genes providing resistance to puromycin, bleomycin, phleomycin, chloramphenicol, Zeocin, and mycophenolic acid. Further selectable markers are described e.g. in WO 92/08796 and WO 94/28143.

A “promoter” refers to a nucleic acid, i.e. polynucleotide sequence, which controls transcription of a nucleic acid to which it is operably linked. A promoter includes signals for RNA polymerase binding and transcription initiation. The promoter(s) used will be functionable in the cell type of the host cell in which expression of the operably linked nucleic acid is contemplated. A large number of promoters including constitutive, inducible, and repressible promoters from a variety of different sources are well known in the art (and identified in databases such as GenBank). They are available as or within cloned polynucleotides (from, e.g., depositories such as ATCC as well as other commercial or individual sources). A “promoter” comprises a nucleotide sequence that directs the transcription of e.g. an operably linked structural gene. Typically, a promoter is located in the 5′ non-coding or 5′-untranslated region (5′UTR) of a gene, proximal to the transcriptional start site of a structural gene. Sequence elements within promoters that function in the initiation of transcription are often characterized by consensus nucleotide sequences. These sequence elements include RNA polymerase binding sites, TATA sequences, CAAT sequences, differentiation-specific elements (DSEs; McGehee, R. E., et al., Mol. Endocrinol. 7 (1993) 551-560), cyclic AMP response elements (CREs), serum response elements (SREs; Treisman, R., Seminars in Cancer Biol. 1 (1990) 47-58), glucocorticoid response elements (GREs), and binding sites for other transcription factors, such as CRE/ATF (O'Reilly, M. A., et al., J. Biol. Chem. 267 (1992) 19938-19943), AP2 (Ye, J., et al., J. Biol. Chem. 269 (1994) 25728-25734), SP1, cAMP response element binding protein (CREB; Loeken, M. R., Gene Expr. 3 (1993) 253-264) and octamer factors (see, in general, Watson, J. D., et al., eds., Molecular Biology of the Gene, 4th ed., The Benjamin/Cummings Publishing Company, Inc. (1987); and Lemaigre, F. P. and Rousseau, G. G., Biochem. J. 303 (1994) 1-14). If a promoter is an inducible promoter, then the rate of transcription increases in response to an inducing agent. In contrast, the rate of transcription is not regulated by an inducing agent if the promoter is a constitutive promoter. Repressible promoters are also known. For example, the c-fos promoter is specifically activated upon binding of growth hormone to its receptor on the cell surface. Tetracycline (tet) regulated expression can be achieved by artificial hybrid promoters that consist e.g. of a CMV promoter followed by two Tet-operator sites. The Tet-repressor binds to the two Tet-operator sites and blocks transcription. Upon addition of the inducer tetracycline, the Tet-repressor is released from the Tet-operator sites and transcription proceeds (Gossen, M. and Bujard, H., Proc. Natl. Acad. Sci. USA 89 (1992) 5547-5551). For other inducible promoters including metallothionein and heat shock promoters, see, e.g., Sambrook, et al. (supra), and Gossen, et al., Curr. Opin. Biotech. 5 (1994) 516-520. Among the eukaryotic promoters that have been identified as strong promoters for high-level expression are the SV40 early promoter, adenovirus major late promoter, mouse metallothionein-I promoter, Rous sarcoma virus long terminal repeat, Chinese hamster elongation factor 1 alpha (CHEF-1, see e.g. U.S. Pat. No. 5,888,809), human EF-1 alpha, ubiquitin, and human cytomegalovirus immediate early promoter (CMV IE). An enhancer (i.e., a cis-acting DNA element that acts on a promoter to increase transcription) may be necessary to function in conjunction with the promoter to increase the level of expression obtained with a promoter alone, and may be included as a transcriptional regulatory element. Often, the polynucleotide segment containing the promoter will include enhancer sequences as well (e.g., CMV or SV40).

“Operably linked” refers to a juxtaposition of two or more components, wherein the components so described are in a relationship permitting them to function in their intended manner. For example, a promoter and/or enhancer is/are operably linked to a coding sequence, if it acts in cis to control or modulate the transcription of the linked coding sequence. Generally, but not necessarily, the DNA sequences that are “operably linked” are contiguous and, where necessary to join two protein encoding regions such as a secretory leader/signal sequence and a polypeptide, contiguous and in reading frame. However, although an operably linked promoter is generally located upstream of the coding sequence, it is not necessarily contiguous with it. Enhancers do not have to be contiguous. An enhancer is operably linked to a coding sequence if the enhancer increases transcription of the coding sequence. Operably linked enhancers can be located upstream, within, or downstream of coding sequences, and at considerable distance from the promoter. A polyadenylation site is operably linked to a coding sequence if it is located at the downstream end of the coding sequence in such a way that transcription proceeds through the coding sequence into the polyadenylation sequence. Linking is accomplished by recombinant methods known in the art, e.g., using PCR methodology, and/or by ligation at convenient restriction sites. If convenient restriction sites do not exist, then synthetic oligonucleotide adaptors or linkers are used in accord with conventional practice.

The term “expression” as used herein refers to transcription and/or translation occurring within a cell. The level of transcription of a desired product in a host cell can be determined on the basis of the amount of corresponding mRNA that is present in the cell. For example, mRNA transcribed from a selected nucleic acid can be quantitated by PCR or by Northern hybridization (see Sambrook, et al., Molecular Cloning: A Laboratory Manual, Cold Spring Harbor Laboratory Press (1989)). The protein encoded by a selected nucleic acid can be quantitated by various methods, e.g. by ELISA, by assaying for the biological activity of the protein, or by employing assays that are independent of such activity, such as Western blotting or radioimmunoassay, by using antibodies that recognize and bind to the protein (see Sambrook et al., 1989, supra).

A “host cell” refers to a cell into which a nucleic acid encoding a heterologous polypeptide is introduced. Host cell includes both prokaryotic cells used for propagation of the nucleic acid (e.g. of plasmids), and eukaryotic cells used for expression of the polypeptide encoded by the nucleic acid (e.g. of a structural gene). Typically, the eukaryotic cells are mammalian cells.

A “polypeptide” is a polymer of amino acid residues joined by peptide bonds, whether produced naturally or synthetically. Polypeptides of less than about 20 amino acid residues may be referred to as “peptides.” Polypeptides comprising two or more amino acid chains or comprising an amino acid chain of a length of 100 amino acids or more may be referred to as “proteins”. A “protein” is a macromolecule comprising one or more amino acid chains whereby in case of one chain this chain has a length of 100 amino acids or more. A polypeptide or protein may also comprise non-peptidic components, such as carbohydrate groups. Carbohydrates and other non-peptidic substituents may be added to a protein by the cell in which the protein is produced, and may vary with the type of cell. Proteins and polypeptides are defined herein in terms of their amino acid backbone structures; additions such as carbohydrate groups are generally not specified, but may be present nonetheless.

“Heterologous DNA” or “heterologous polypeptide” refers to a DNA molecule or a polypeptide, or a population of DNA molecules or a population of polypeptides, that do not exist naturally within a given host cell. DNA molecules heterologous to a particular host cell may contain DNA derived from the host cell species (i.e. endogenous DNA) so long as that host cell derived DNA is combined with non-host cell derived DNA (i.e. exogenous DNA). For example, a DNA molecule containing a non-host DNA segment encoding a polypeptide operably linked to a host DNA segment comprising a promoter is considered to be a heterologous DNA molecule. Conversely, a heterologous DNA molecule can comprise an endogenous structural gene operably linked with an exogenous promoter. A peptide or polypeptide encoded by a non-host DNA molecule is a “heterologous” peptide or polypeptide.

A “cloning plasmid” is a nucleic acid molecule, such as a vector, cosmid, phagemid or bacterial artificial chromosome (BAC), which has the capability of replicating autonomously in a host cell. Cloning plasmids typically contain one or a small number of restriction endonuclease recognition sites that e.g. allow the insertion of a nucleic acid in a determinable fashion without loss of an essential biological function of the plasmid, as well as nucleotide sequences providing a selectable marker that is suitable for use in the identification and selection of cells transformed with the cloning plasmid. Selectable markers typically include genes that provide tetracycline, puromycin, hygromycin, or ampicillin resistance.

An “expression plasmid” is a nucleic acid molecule encoding a polypeptide to be expressed in a host cell. Typically, an expression plasmid comprises a prokaryotic plasmid propagation unit, e.g. for E. coli, which comprises an origin of replication, and a selectable marker, an eukaryotic selectable marker, and one or more expression cassettes for the expression of the nucleic acid(s) of interest each comprising a promoter, a structural gene, and a transcription terminator including a polyadenylation signal. Gene expression is usually placed under the control of a promoter, and such a structural gene is said to be “operably linked to” the promoter. Similarly, a regulatory element and a core promoter are operably linked if the regulatory element modulates the activity of the core promoter.

The term “immunoglobulin” refers to a protein consisting of one or more polypeptide(s) substantially encoded by immunoglobulin genes. The recognized immunoglobulin genes include the different constant region genes as well as the myriad immunoglobulin variable region genes. Immunoglobulins may exist in a variety of formats, including, for example, Fv, Fab, and F(ab)₂ as well as single chains (scFv) or diabodies (e.g. Huston, J. S., et al., PNAS USA 85 (1988) 5879-5883; Bird, R. E., et al., Science 242 (1988) 423-426; in general, Hood, L. E., et al., Immunology, Benjamin N.Y., 2nd edition (1984); and Hunkapiller, T. and Hood, L. E., Nature 323 (1986) 15-16).

An immunoglobulin in general comprises two so called light chain polypeptides (light chain) and two so called heavy chain polypeptides (heavy chain). Each of the heavy and light chain polypeptides contains a variable domain (variable region, generally the amino terminal portion of the polypeptide chain) comprising binding regions that are able to interact with an antigen. Each of the heavy and light chain polypeptides comprises a constant region (generally the carboxyl terminal portion). The constant region of the heavy chain mediates the binding of the antibody i) to cells bearing a Fc gamma receptor (FcγR), such as phagocytic cells, or ii) to cells bearing the neonatal Fc receptor (FcRn) also known as Brambell receptor. It also mediates the binding to some factors including factors of the classical complement system such as component (Clq).

The variable domain of an immunoglobulin's light or heavy chain in turn comprises different segments, i.e. four framework regions (FR) and three hypervariable regions (CDR).

An “immunoglobulin fragment” denotes a polypeptide comprising at least one domain of the group of domains comprising the variable domain, the C_{H}1 domain, hinge-region, the C_{H}2 domain, the C_{H}3 domain, the C_{H}4 domain of a heavy chain of an immunoglobulin or the variable domain or the C_{L} domain of a light chain of an immunoglobulin. Also comprised are derivatives and variants thereof. Additionally a variable domain, in which one or more amino acids or amino acid regions are deleted, may be present.

An “immunoglobulin conjugate” denotes a polypeptide comprising at least one domain of an immunoglobulin heavy or light chain conjugated via a peptide bond to a further polypeptide. The further polypeptide is a non-immunoglobulin peptide, such as a hormone, growth receptor, antifusogenic peptide or the like.

A “mitogenic receptor” is a receptor that if activated or deactivated positively or negatively affects cell division, i.e. cell growth. The term “positively affects cell division” denotes that the mitogenic receptor promotes cell division. Preferably the mitogenic receptor is a transmembrane receptor with an intracellular phosphorylation site. If this phosphorylation site is phosphorylated the receptor activates cell proliferation. A preferred mitogenic receptor is the chimeric ErbB2/IGF-I receptor.

The term “constitutively active” denotes that a receptor is in its active form, i.e. positively affects cell division, without the need of a further binding partner, i.e. activation signal. This can be achieved e.g. with the introduction of a mutation in the receptor's amino acid sequence. A preferred constitutively active mitogenic receptor is the chimeric ErbB2^{V→E}/IGF-I receptor.

Insulin-like growth factor I receptor (IGF-IR, EC 2.7.1.112, CD 221 antigen, also denoted as IGF-IR) belongs to the family of transmembrane protein tyrosine kinases (LeRoith, D., et al., Endocrin. Rev. 16 (1995) 143-163; Adams, T. E., et al., Cell. Mol. Life. Sci. 57 (2000) 1050-1093). IGF-IR binds IGF-I with high affinity and initiates the physiological response to this ligand in vivo. IGF-IR also binds to IGF-II, however with slightly lower affinity. The activation of IGF-IR, e.g. by binding IGF-I (also generally denoted as effector or ligand in the following), triggers cellular signaling cascades which may result in mitogenic as well as metabolic effects (see e.g. Humbel, R. E., Eur. J. Biochem. 190 (1990) 445-462). The binding of an effector to IGF-IR results in the activation of a tyrosine kinase. With point-mutations in growth factor receptors a constitutive activation of the receptor tyrosine kinase can be achieved, e.g. V664E Neu or V664Q Neu (Bargmann, C. I., et al., Cell 45 (1986) 649-657), V922E IGF-IR (Takahashi, K., et al., J. Biol. Chem. 270 (1995) 19041-19045), L301S CSF-1R or Y969F CSF-1R (Roussel, M. F., Cell 55 (1988) 979-988), C332Y FGFR-2 (Neilson, K. M. and Friesel, R. E., J. Biol. Chem. 270 (1995) 26037-26040), V938D IR (Longo, N., J. Biol. Chem. 267 (1992) 12416-12419), V560G c-Kit or D814V c-Kit (Furitsa, T., et al., J. Clin. Invest. 92 (1993) 1736-1744). E.g. the V922E mutation in IGF-IR results in a constitutively augmented tyrosine kinase activity of mutated IGF-IR. CHO cells expressing V922E IGF-IR showed significantly stimulated glucose uptake but did not promoted mitogenesis in the absence of IGF-I.

Mitogenic activity of a cell can be determined e.g. by thymidine uptake assay, or cell proliferation assay with XTT (see e.g. Scudiero, D. A., et al., Cancer Res. 48 (1988) 4827-4833).

The site of modification of a mitogenic receptor in order to become a constitutively active mitogenic receptor can be, e.g., in the transmembrane-strech. The introduced modification may result in a conformational change of the intracellular kinase-domain resulting in a constitutive activation of the domain. This constitutive activation provides for a ligand-independent activation of the modified receptor. If such a mitogenic receptor is constitutively active it provides the cell comprising it an enhanced ability to proliferate. This proliferation ability is in the presence of such a constitutively active mitogenic receptor independent of the presence of the ligand of the not modified receptor, which would normally activate the mitogenic properties. In one embodiment the cell according to the invention is growing in suspension. In another embodiment is the cell according to the invention adapted to growth in serum free medium. A cell providing a constitutively active mitogenic receptor is independent of the corresponding ligand of the receptor for growth, i.e. it can grow even in the absence of the growth promoting ligand. A constitutively active mitogenic receptor possesses an extracellular domain, a transmembrane-strech, a tyrosine-kinase domain directly following the transmembrane-strech, and a hydrophilic intracellular domain. Said receptor has still the property of binding its activating ligand.

One embodiment of the current invention comprises a constitutively active mitogenic receptor which is a fusion polypeptide comprising the extracellular and transmembrane domains of the ErbB2 receptor (amino acids (AA) 1-682 of the ErbB2 receptor, for human ErbB2 receptor see SEQ ID NO: 09) with the mutation V659E and the cytoplasmic domain of the IGF-I receptor β-subunit (AA 929-1337 of the IGF-I receptor, for human IGF-I receptor see SEQ ID NO: 10) expressed in a CHO cell. The term “AA” is used for “amino acid position”.

The term “amino acid” as used within this application denotes a group of carboxy α-amino acids, which directly or in form of a precursor can be encoded by a nucleic acid. The group of amino acids comprises alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

Thus, one aspect of the current invention is a CHO cell, which is expressing a constitutively active mitogenic receptor. In one embodiment of the this aspect is the CHO cell comprising a nucleic acid encoding the extracellular and transmembrane domains of the ErbB2 receptor (amino acids (AA) 1-682) with the mutation V659E and the cytoplasmic domain of the IGF-I receptor 8-subunit (AA 929-1337) with the amino acid sequence shown in SEQ ID NO: 03. In another embodiment of the current invention is the CHO cell a CHO-K1 cell, or a CHO-DHFR⁻-cell (DSMZ ACC 126), or a CHO DG44-cell, preferably a CHO-K1 cell.

The second aspect of the current invention is the cell line CHO-K1 ErbB2V>E/IGF-I DSM ACC2851.

Another aspect of the current invention is a method for obtaining a CHO cell according to the invention with a constitutively active mitogenic receptor, wherein the method comprises the following steps:
a) transfecting a CHO cell with a first nucleic acid comprising
   i) an expression cassette comprising a nucleic acid encoding a selectable marker flanked by two loxP-sites, one in 3′ position (downstream) and one in 5′ position (upstream) of the expression cassette,
   ii) optionally an expression cassette comprising a nucleic acid encoding transferrin,
   iii) an expression cassette comprising a nucleic acid encoding a constitutively active mitogenic receptor,
b) selecting a CHO cell transfected with the first nucleic acid,
c) transfecting the CHO cell selected in step b) with a second nucleic acid comprising a CRE recombinase expression cassette,
d) selecting a CHO cell transfected with the second nucleic acid as a CHO cell with a constitutively active mitogenic receptor.

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The loxP-CRE recombinase system is a site-specific recombination system, wherein the loxP-sites define the sites of recombination and the CRE recombinase catalyzes the recombination of the nucleic acid (Sternberg, N. and Hamilton, D., J. Mol. Biol. 150 (1981) 467-486; Abremski, K. and Hoess, R. E., Gene 25 (1983) 49-58; Hoess, R. E., and Abremski, K., J. Mol. Biol. 181 (1985) 351-362).

In one embodiment of the current aspect of the invention expresses the transfected CHO cell of step b) a constitutively active mitogenic receptor. In a preferred embodiment comprises the constitutively active mitogenic receptor the extracellular and transmembrane domains of the ErbB2 receptor (amino acids (AA) 1-682) with the mutation V659E and the cytoplasmic domain of the IGF-I receptor β-subunit (AA 929-1337). In another preferred embodiment has the constitutively active mitogenic receptor the amino acid sequence of SEQ ID NO: 03.

The CHO cell according to the invention is a CHO cell with a constitutively active mitogenic receptor and, in another embodiment the transfected CHO cell is selected in step d) for the absence of the selectable marker comprised in the first nucleic acid. In still another embodiment the transfected CHO cell selected in step d) is not growing in the presence of a selection agent according to the selectable marker of the first nucleic acid, i.e. for which the first nucleic acid provides resistance. It has now surprisingly been found that a CHO-K1 cell according to the invention expressing a constitutively active chimeric ErbB2^{V→E}/IGF-I mitogenic receptor has improved growth characteristics. In one embodiment said CHO cell grows in cultivation to a maximum cell density of at least 5×10⁶ cells/ml. In a preferred embodiment said CHO cell grows in cultivation to a maximum cell density of at least 8×10⁶ cells/ml. In a further embodiment said maximum cell density is reached within 8 to 12 generations starting from a cell density of approximately 2 to 3×10⁵ cells/ml in a volume of 60 to 70 ml. In another embodiment the cultivation of said CHO cell is a fed-batch cultivation. In one embodiment the cell density reached by said CHO cell is over at least 5 generations not less than 95% of the cell density reached after 6 generations of cultivation as fed-batch cultivation. In another embodiment the cell density reached by said CHO cell is over at least 6 generations not less than 75% of the cell density after 6 generations of cultivation as fed-batch cultivation. In a further embodiment said cell density after 6 generations of cultivation is reached starting from a cell density of approximately 2 to 3×10⁵ cells/ml in a volume of 60 to 70 ml. The cell density is determined by CASY as reported in the examples.

A forth aspect of the current invention is a method for the recombinant production of a heterologous polypeptide in a CHO cell according to the invention which is expressing a constitutively active mitogenic receptor. In one embodiment the heterologous polypeptide is a biologically active polypeptide.

The term “biologically active polypeptide” as used herein refers to an organic molecule, e.g. a biological macromolecule such as a peptide, protein, glycoprotein, nucleoprotein, mucoprotein, lipoprotein, synthetic polypeptide or protein, that causes a biological effect when administered in or to artificial biological systems, such as bioassays using cell lines and viruses, or in vivo to an animal, including but not limited to birds or mammals, including humans. This biological effect can be, but is not limited to, enzyme inhibition or activation, binding to a receptor or a ligand, either at the binding site or circumferential, signal triggering or signal modulation. Biologically active molecules are without limitation for example immunoglobulins, or hormones, or cytokines, or growth factors, or receptor ligands, or agonists or antagonists, or cytotoxic agents, or antiviral agents, or imaging agents, or enzyme inhibitors, enzyme activators or enzyme activity modulators such as allosteric substances. In one embodiment the heterologous polypeptide is an immunoglobulin, immunoglobulin conjugate, or an antifusogenic peptide.

An “antifusogenic peptide” is a peptide which inhibits events associated with membrane fusion or the membrane fusion event itself, including, among other things, the inhibition of infection of uninfected cells by a virus due to membrane fusion. These antifusogenic peptides are preferably linear peptides. For example, they can be derived from the gp41 ectodomain, e.g. such as DP107, DP178. Examples of such peptides can be found in U.S. Pat. No. 5,464,933, U.S. Pat. No. 5,656,480, U.S. Pat. No. 6,013,263, U.S. Pat. No. 6,017,536, U.S. Pat. No. 6,020,459, U.S. Pat. No. 6,093,794, U.S. Pat. No. 6,060,065, U.S. Pat. No. 6,258,782, U.S. Pat. No. 6,348,568, U.S. Pat. No. 6,479,055, U.S. Pat. No. 6,656,906, WO 1996/19495, WO 1996/40191, WO 1999/59615, WO 2000/69902, and WO 2005/067960. For example, the amino acid sequences of such peptides comprise SEQ ID NO: 1 to 10 of U.S. Pat. No. 5,464,933; SEQ ID NO: 1 to 15 of U.S. Pat. No. 5,656,480; SEQ ID NO: 1 to 10 and 16 to 83 of U.S. Pat. No. 6,013,263; SEQ ID NO: 1 to 10, 20 to 83 and 139 to 149 of U.S. Pat. No. 6,017,536; SEQ ID NO: 1 to 10, 17 to 83 and 210 to 214 of U.S. Pat. No. 6,093,794; SEQ ID NO: 1 to 10, 16 to 83 and 210 to 211 of U.S. Pat. No. 6,060,065; SEQ ID NO: 1286 and 1310 of U.S. Pat. No. 6,258,782; SEQ ID NO: 1129, 1278-1309, 1311 and 1433 of U.S. Pat. No. 6,348,568; SEQ ID NO: 1 to 10 and 210 to 238 of U.S. Pat. No. 6,479,055; SEQ ID NO: 1 to 171, 173 to 216, 218 to 219, 222 to 228, 231, 233 to 366, 372 to 398, 400 to 456, 458 to 498, 500 to 570, 572 to 620, 622 to 651, 653 to 736, 739 to 785, 787 to 811, 813 to 815, 816 to 823, 825, 827 to 863, 865 to 875, 877 to 883, 885, 887 to 890, 892 to 981, 986 to 999, 1001 to 1003, 1006 to 1018, 1022 to 1024, 1026 to 1028, 1030 to 1032, 1037 to 1076, 1078 to 1079, 1082 to 1117, 1120 to 1176, 1179 to 1213, 1218 to 1223, 1227 to 1237, 1244 to 1245, 1256 to 1268, 1271 to 1275, 1277, 1345 to 1348, 1350 to 1362, 1364, 1366, 1368, 1370, 1372, 1374 to 1376, 1378 to 1379, 1381 to 1385, 1412 to 1417, 1421 to 1426, 1428 to 1430, 1432, 1439 to 1542, 1670 to 1682, 1684 to 1709, 1712 to 1719, 1721 to 1753, 1755 to 1757 of U.S. Pat. No. 6,656,906; or SEQ ID NO: 5 to 95 of WO 2005/067960. In one embodiment the antifusogenic peptide has an amino acid sequence comprising of from 5 to 100 amino acids, preferably of from 10 to 75 amino acids and more preferred of from 15 to 50 amino acids.

In another embodiment the heterologous polypeptide is an immunoglobulin, or an immunoglobulin fragment, or an immunoglobulin conjugate.

This aspect of the current invention comprises a method for the recombinant production of a heterologous polypeptide in a cell according to the invention. The cell for the recombinant production comprises a third nucleic acid comprising an expression cassette for the expression of the heterologous polypeptide. This third nucleic acid has been introduced into the cell on e.g. an expression plasmid and the cell is cultivated under conditions suitable for the expression of the heterologous polypeptide. Thus, another aspect of the current invention is a method for the production of a heterologous polypeptide, wherein the method comprises the following steps:
a) providing a CHO-K1 cell expressing a constitutively active chimeric ErbB2^{V→E}/IGF-I mitogenic receptor,
b) transfecting said cell with a nucleic acid comprising an expression cassette for the expression of a heterologous polypeptide,
c) recovering said heterologous polypeptide from the cell or the cultivation medium.

In one embodiment said heterologous polypeptide is an immunoglobulin, or an immunoglobulin fragment, or an immunoglobulin conjugate.

Immunoglobulin molecules are assigned to five different classes: IgA (Immunoglobulin A), IgD, IgE, IgG, and IgM. Of these IgG and IgE are more frequently used in pharmaceutic and diagnostic applications. Within these classes the immunoglobulins differ in their over-all structure but the building blocks are similar. All immunoglobulins are built up of two different polypeptide chains, a light chain and a heavy chain.

An “immunoglobulin fragment” comprises the carboxy-terminal constant domain(s) of an immunoglobulin light or heavy chain, e.g. it comprises either at least the C_{H}1-, C_{H}2-, C_{H}3-domain and the hinge-region of an immunoglobulin heavy chain and optionally a C_{H}4-domain, or the C_{L}-domain of an immunoglobulin light chain. The immunoglobulin from which the fragment is derived can be a naturally occurring or a synthetic immunoglobulin, or a rodent immunoglobulin, or a humanized immunoglobulin, or a human immunoglobulin. In one embodiment of the invention the immunoglobulin fragment additionally contains a fragment of a heavy or light chain variable domain or of a variant thereof. In the variable domain fragment amino acid(s) or region(s) are deleted. In one embodiment of from one to six amino acids of the variable domain are deleted. In another embodiment of from one to six regions of the variable domain are deleted. In a further embodiment the variable domain is deleted. In one embodiment, the variable and constant domains comprised in the immunoglobulin fragment are of/derived from the same antibody, i.e. belong to the same antibody.

For expression the nucleic acid encoding the heterologous polypeptide is introduced into an expression plasmid comprising all required elements for the expression of the heterologous polypeptide in a CHO cell according to the invention in an operably linked form.

In one embodiment the CHO cell comprises a nucleic acid encoding a heterologous polypeptide. In one embodiment the heterologous polypeptide is a human polypeptide. In another embodiment the polypeptide is selected from immunoglobulins, or immunoglobulin heavy chains, or immunoglobulin light chains, or immunoglobulin fragments, or immunoglobulin conjugates.

The preferred cell line according to the invention, cell line CHO-K1 ErbB2V>E/IGF-I was deposited, under the Budapest Treaty on the international recognition of the deposit of microorganisms for the purposes of patent procedure, with Deutsche Sammlung von Mikroorganismen and Zellkulturen GmbH (DSMZ), Germany, on Jun. 13, 2007 under Accession No. DSM ACC2851.

The following examples, sequence listing and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 Plasmid map of plasmid 5519-pUC.

FIG. 2 Plasmid map of plasmid 4699-pUC-Hyg.

FIG. 3 Plasmid map of plasmid pMC-CRE.

FIG. 4 Cell density during the cultivation of CHO-K1 adapted to suspension culture, CHO-K1-5519-1B6-18B3 and CHO-K1-5519-1B6-17C5; X-Axis: days; Y-Axis: cell density in 10⁶ cells/ml; filled squares: CHO-K1 adapted to suspension growth, filled circles: CHO-K1-5519-1B6-18B3, filled triangles: CHO-K1-5519-1B6-17C5.

### EXAMPLES

The CHO cell line according to the invention was generated by 3 successive complete best clone selection campaigns beginning with a parental CHO-K1(W) cell line, which is a CHO-K1 cell line (ATCC CCL-61) adapted to growth in suspension culture.

### Example 1

### Generation of Plasmid 5519-pUC

The plasmid 5519-pUC provides expression cassettes for a constitutively active mitogenic receptor (the chimeric receptor ErbB2^{V→E}/IGF-IR), an expression cassette for the expression of transferrin, and an expression cassette for a selectable marker conferring resistance to puromycin.

In detail, plasmid 5519-pUC comprises of the following elements:
a nucleic acid comprising the puromycin selectable marker flanked by two loxP-sites (SEQ ID NO: 01); this nucleic acid is designated in the following as puro-loxP.
an pUC-origin of replication for replication and growth of the plasmid in E. coli,
a beta-lactamase gene (β-lactamase gene),
a nucleic acid comprising the transferrin structural gene, a signal peptide, the SV40 early promoter and origin, and the cDNA-sequence of the transferrin-open reading frame (Yang, F., et al., Proc. Natl. Acad. Sci. USA 81 (1984) 2752-6) (SEQ ID NO: 02),
a nucleic acid comprising the SV40 early promoter- and origin-sequence, and a cDNA-sequence encoding the extracellular and transmembrane domains of the ErbB2 receptor (amino acids (AA) 1-682) with the mutation V659E and the cytoplasmic domain of the IGF-I receptor 8-subunit (AA 929-1337) (Belaus, A., et al., J. Steroid. Biochem. Mol. Biol. 85 (2003) 105-15) (SEQ ID NO: 03). This chimeric receptor is designated as ErbB2^{V→E}/IGF-IR.

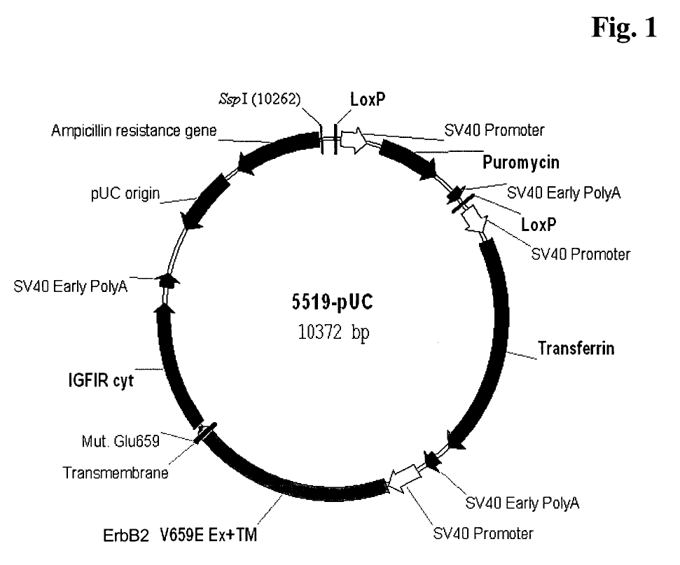

The elements of plasmid 5519-pUC were introduced in the vector 4699-pUC-Hyg. An annotated plasmid map of plasmid 5519-pUC is shown in FIG. 1.

The vector 4699-pUC-Hyg was obtained by PCR-amplification of a 3860 by DNA fragment starting from nucleotide 1731 to nucleotide 5590 of the vector pcDNA3.1/Hygro (+) (Cat-No.: V870-20, Invitrogen Corp., USA). An additional 56 by nucleic acid fragment was introduced by the PCR-Primers (forward primer: ataatacctgcaggaaaaggccggccaaaggatccctgtggaatgtgtgtcagttagggtg (SEQ ID NO: 11); reverse primer: ttattcctgcaggtattatcaccggtgattggcgcgccaggtggcacttacggggaaatgtg (SEQ ID NO: 04)) containing the AscI, SgrAI, AscI, Sbfl, and BamHI restriction sites, relegation of the resulting PCR product occurred within the Sse383871 restriction site, resulting in the circulated plasmid 4699-pUC-Hyg comprising 3916 bp.

The plasmid 4699-pUC-Hyg comprises of the following elements:
a nucleic acid comprising the hygromycin selectable marker (hyg),
an pUC-origin of replication for replication and growth of the plasmid in E. coli,
a beta-lactamase gene.

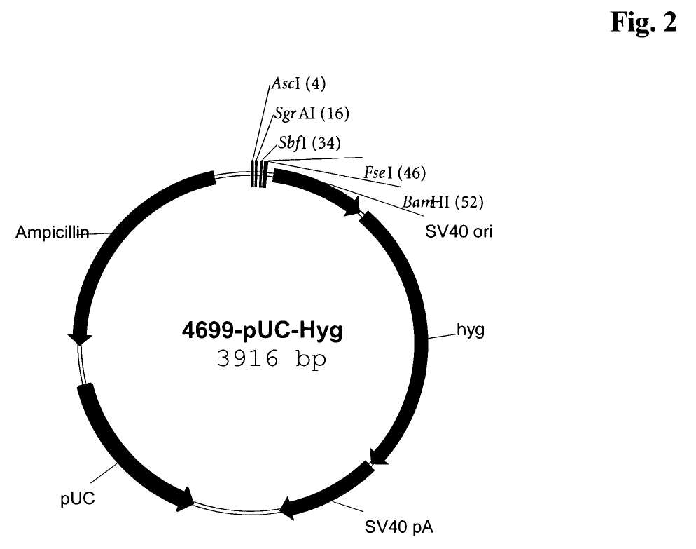

An annotated plasmid map of plasmid 4699-pUC-Hyg is shown in FIG. 2.

### Example 2

### Transfection of CHO-K1 Cells with Plasmid 5519-pUC

In order to obtain a CHO cell with a constitutively active mitogenic receptor, CHO-K1 cells were adapted to growth in serum-free media. The basic CHO-K1 cell line was obtained from the American Type Culture Collection (ATCC CCL-61). The derivation and creation of this CHO-K1 cell line is described by Kao, F. T. and Puck, T. T., Proc. Natl. Acad. Sci. USA 60 (1968) 1275-81; and Puck, T. T., et al., J. Exp. Med. 108 (1958) 945-56.

The adapted cell line was named CHO-K1(W). This cell line does not contain extraneous DNA, e.g. integrated by transfection methods, and is adapted to growth in suspension culture in synthetic, animal component-free ProCHO4 medium (Cambrex Corp., USA). This medium is supplemented with 8 mM glutamine (Gln) and 1×HT (Hypoxanthine-Thymidine) supplement and is designated in the following as ProCHO4-complete medium.

Plasmid 5519-pUC was linearized prior to transfection using the single SspI-restriction site in proximity to the ampicillin selectable marker (gene). CHO-K1(W) cells were electroporated with the linearized DNA using the Gene Pulser XCell electroporation device (Bio-Rad Laboratories GmbH, Germany) with 2 mm gap electroporation cuvettes. The electroporation pulse used was 160 V/15 ms applied on 20 μg of plasmid DNA and 7.5×10⁶ cells in a total volume of 200 μl of Dulbecco's PBS (Cat No: D8537, Sigma-Aldrich GmbH, Seelze, Germany). Afterwards the cells were resuspended in ProCHO4-complete medium and plated into twenty 96-well multiwell plates at 5000 cells/well. After 24 hours the growth medium was changed to ProCHO4-complete selection medium (ProCHO4-complete medium supplemented with 5 μg/ml Puromycin).

### Example 3

### Generation of Cell Line CHO-K1-5519-1B6

The transfected cells of Example 2 were cultivated for 2-4 weeks in ProCHO4-complete selection medium. To obtain a stably transfected cell clone two selection steps were performed.

### a) First Selection Step:

Two different criteria were used to pick clones for propagation:

### Visual Selection

Visual predominant appearing clones were transferred to 24-well multiwell plates (non-tissue culture treated; Becton Dickinson, Surface area: 2.0 cm²).

### Cell Proliferation Assay

A WST-I cell proliferation assay (Roche Diagnostics GmbH, Germany) was performed for selection of additional clones according to growth parameters. The clones with the highest absorbance were selected and transferred to the 24-well multiwell plates.

The cell proliferation and viability of clones was determined with the WST-I colorimetric assay. The assay is based on the direct correlation of the metabolic activity of cells to the increasing amount of accumulated formazan-dye, which is produced by the cleavage of the WST-I reagent by mitochondrial dehydrogenases.

For the assay cells were duplicated. One aliquot was splitted 1:10 to a final volume of 100 μl into a well of 96-well multiwell plate. After a cultivation time of 24 hours 10 μl of WST-I cell proliferation reagent were added to each well. Thereafter the cells were incubated for an additional 2 hours at 37° C./5% CO₂. The absorbance of the samples was measured using a Tecan reader (Spectrafluorplus, Tecan Deutschland GmbH, Germany) at 450 nm with a reference wavelength of 620 nm.

### b) Second Selection Step

For further clone selection in 24-well-multiwell plate, 6-well multiwell plate, and finally shaker flask format a defined seeding strategy was used. Therefore 3×10⁵ cells/ml were seeded in the respective cell culture vessel and the cell density and viability after 4 and 7-8 days was determined using a CASY cell counter (Schärfe Systems, Reutlingen, Germany). Cell clumps were dissolved by incubating 200 μl of the cell suspension with 20 μl Trypsin for 30 min. at 37° C. For measurement 50 or 100 μl of the disaggregated cell suspension was diluted in 10 ml CASYton-buffer (Schärfe Systems GmbH, Germany) and the total viable cell-concentration in 400 μl thereof was determined in triplicate by electronic pulse area analysis using the Cell Counter and Analyzer-System CASY (Schärfe System GmbH, Germany). The result of a measurement using a 150 μm capillary is a size distribution curve. The size distribution is classified for three types of particles: 3.4-5 μm as cell debris; 5-10 μm as dead cells; 10-30 μm as viable cells.

The cell clones with highest cell densities after 4 days (indicating fast proliferation) and 7-8 days were identified and expanded. Exemplary results are shown in Table 1.

TABLE 1
Exemplary results of the CASY selection of cultivations
in 125 ml shaker flasks under batch culture conditions
(total volume: 62.5 ml in ProCHO4-complete medium).

| | Cell | Cell | Cell | Cell |
|---|---|---|---|---|
| | count/ml | viability | count/ml | viability |
| Cell clone | Day 4 | Day 4 | Day 8 | Day 8 |
| CHO-K1-5519-1B6 | 5.5 × 10⁶ | 99% | 8.8 × 10⁶ | 99% |
| CHO-K1-5519-1C1 | 5.5 × 10⁶ | 99% | 3.5 × 10⁶ | 68% |
| CHO-K1-5519-1C4 | 3.0 × 10⁶ | 97% | 2.0 × 10⁶ | 94% |
| CHO-K1-5519-1E3 | 2.7 × 10⁶ | 97% | 2.0 × 10⁶ | 92% |
| CHO-K1-5519-1G5 | 4.9 × 10⁶ | 99% | 7.6 × 10⁶ | 98% |

The transfectant mixed cell clone CHO-K1-5519-1B6 with best growth properties was selected after first round of clone selection.

### Example 4

### Generation of Cell line CHO-K1 (PuDL)

A single cell deposition step by flow cytometry (Fluorescence Activated Cell Sorting, FACS) was used for the generation of cell line CHO-K1(PuDL).

The transfectant mixed cell clone CHO-K1-5519-1B6 with best growth properties selected in Example 3 was cultivated in ProCHO4-complete medium with 4 μg/ml puromycin. 1×10⁷-cells in logarithmic growth phase were harvested, disaggregated by passing through a 40 μm filter to eliminate cell clumps (BD Falcon; cell strainer, BD Biosciences, USA), pelleted by centrifugation (10 min. at 300×g) and resuspended in 5 ml of ProCHO4-complete medium to obtain a solution with 1-2×10⁶ cells/ml without the presence of a selecting agent in the medium. Using a FACSAria cell sorter (BD Biosciences, USA) a single cell was deposited into each well of twenty 96-well multiwell plates (U-shape, Cellstar, Greiner bio-one, Frickenhausen, Germany) containing 50 μl cell-conditioned ProCHO4-complete medium and 50 μl fresh ProCHO4-complete medium without a selecting agent. Two days after the single cell deposition step, 100 μl of double-concentrated selective ProCHO4-complete medium containing 8 μg/ml Puromycin was added. The 82 first appearing subclones were expanded after 17 days into 24-well multiwell plates.

For the further clone selection in 24-well-multiwell plates, 6-well multiwell plates, and shaker flasks the previously described defined seeding strategy was used (see Example 3). The cell clones with highest cell densities after 4 days and 7-8 days, respectively, were identified and expanded. The clone CHO-K1-5519-1B6-18B3, hereinafter denoted as CHO-K1 (PuDL), was selected based on his growth characteristics after growth in fed-batch suspension culture (125 ml Erlenmeyer flasks) in ProCHO4-complete medium.

### Example 5

### Transient Transfection of Cell Line CHO-K1 (PuDL) with a CRE-Recombinase Expression Plasmid

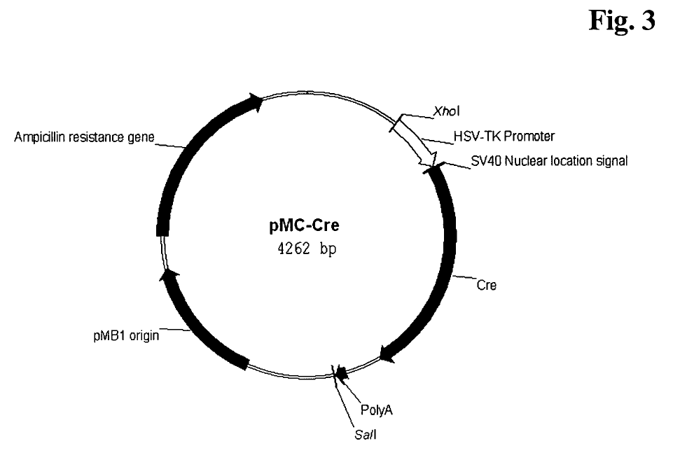

The construction of the CRE-recombinase expression plasmid pMC-CRE was described by Gu et al. (Gu, H., et al. Cell 73 (1993) 1155-1164). It contains the CRE coding part together with the promoter/enhancer and pA regions from pMC1NeopA. For an annotated plasmid map see FIG. 3.

For transfection CHO-K1(PuDL) cells of Example 4 were electroporated with circular plasmid DNA using the Gene Pulser XCell electroporation device (Bio-Rad Laboratories Inc., USA) according to the manufacturer's instruction. Two transfection approaches were pooled and resuspended in 37.5 ml ProCHO4-complete medium with a density of 4×10⁵ cells/ml.

### Example 6

### Selection of Cell Line CHO-K1 ErbB2V>E/IGF-I

The cells of Example 5 were cultivated for 7 generations (24 days) in ProCHO4-complete medium without selection agent prior to a single cell deposition step by flow cytometry.

For this selection the FACSAria cell sorter (BD Biosciences) was used to deposit a single cell into each well of twenty 96-well multiwell plates (U-shape, Cellstar, Greiner Bio-One GmbH, Frickenhausen, Germany) containing 50 μl cell-conditioned ProCHO4-complete medium and 50 μl fresh ProCHO4-complete medium. Three days after the single cell deposition step, 100 μl of fresh ProCHO4-complete medium was added. After 15 days of cultivation the 127 first appearing subclones were expanded into 24-well multiwell plates. For the further clone selection the previously described defined seeding strategy was used (Example 3). Thirty-one cell clones with highest cell densities after 9 days were identified using the CASY Cell Counter-System.

Clone CHO-K1 ErbB2V>E/IGF-I was selected based on his growth characteristics after growth in 125 ml fed-batch suspension culture in ProCHO4-complete medium. Clone CHO-K1 ErbB2V>E/IGF-I has been deposited with DSMZ as CHO-K1 ErbB2V>E/IGF-I on Jun. 13, 2007 as DSM ACC2851. Furthermore Clone CHO-K1 ErbB2V>E/IGF-I was characterized to be sensitive for the selection marker Puromycin (5 μg/ml), which was confirmed by RT-PCR analyses verifying the absence of pac-mRNA transcripts in CHO-K1 ErbB2V>E/IGF-I cells.

### Cell Proliferation Assay

The WST-I colorimetric assay as described above was used for the indirect determination of successful removal of the functional nucleic acid puro-loxP. Therefore cells of each subclone were resuspended in ProCHO4-complete medium either containing no or 5 μg/ml puromycin, respectively. Both cell suspensions of each subclone were plated in triplicate with a concentration of 3×10³ cells/100 μl into five 96-well multiwell plates. For a time period of five days, 10 μl of WST-I Cell proliferation reagent/well was added each day to one triplicate set containing 5 μg/ml puromycin or not. Cells were incubated for 2 hours at 37° C./5% CO₂ and absorbance was measured using a Tecan reader (Spectrafluorplus) at 450 nm with a reference wavelength of 620 nm. Inhibition of cell proliferation in puromycin-containing ProCHO4-complete medium compared to puromycin-free medium revealed 17 of 31 examined subclones to be puromycin-sensitive.

Detection of Puromycin N-acetyl Transferase mRNA-Molecules in Subclones with RT-PCR

For the detection of puromycin N-acetyl transferase mRNA in selected subclones a hybridization probe RT-PCR-assay was developed using the LightCycler Instrument (Roche Diagnostics GmbH, Germany).

### RNA-Isolation:

Total RNA was isolated from cells grown in 6-well multiwell-plates using the RNeasy mini kit (Qiagen, Hilden, Germany). The extraction was performed according to the manufacturer's protocol. Traces of contaminating DNA was subsequently removed using the TURBO DNA-free Kit (Ambion Inc., Austin, Tex., USA) according to the manufacturer's protocol. Quantification of RNA samples and purity of RNA was determined by measuring the absorbance at 260 nm and 280 nm in a UVIKON 931 spectrophotometer (Kontron Instruments, Italy).

Hybridization Probe Assay for pac-mRNA Detection Using Real-Time RT-PCR:

Isolated total RNA of each cell clone was reversely transcribed and subsequently amplified in a LightCycler apparatus using the one-step-RT-PCR LightCycler RNA Hybprobe kit (Roche Diagnostics GmbH, Germany) according to the manufacturer's protocol. The “Second Derivative Maximum Method” was used to determine the Crossing point (Cp) for the individual samples. The employed primer pair was: pacF 5′-AGCTGCAAGAACTCTTCCTCAC-3′ (SEQ ID NO: 5), and pacR 5′-TCAGGCACCGGGCTT-3′ (SEQ ID NO: 6) amplifying a 457 by fragment of the pac-mRNA. Hybprobe probes were designed and Fluorescein- or LightCycler Red 640 labeled at TIB MOLBIOL (Berlin, Germany). The employed set of Hybprobes was pacFL: CCCGCCTTCCTGGAGACCTCC-FL (SEQ ID NO: 7) labeled with fluorescein at its 3′-end and pacLC: 640-CGCCCCGCAACCTCCCCT-p (SEQ ID NO: 8) labeled at its 5′-end with LC Red 640 dye. The free 3′-hydroxy group of pacLC oligonucleotide probe was blocked with a phosphate. To ensure correct product amplification all samples were separated by 2% (w/w) agarose-gel electrophoresis after PCR.

Reaction mixture: A total volume of 20 μl contained RNA Master Hybprobe mix, 500 nM (each) forward and reverse primer (pacF and pacR), 200 nM of each probe (pacFL and pacLC), 3.25 mM of Mn(OAc)₂, and 100 nanogram of total RNA. RT-PCR conditions were as follows: RT at 61° C. for 20 min. and 95° C. for 2 min., 45 cycles of PCR with 95° C. for 4 sec., 55° C. for 15 sec. and 72° C. for 20 sec.

### Example 7

### Detection of ErbB2^{v→E}/IGF-IR Protein-Expression

### Protein Extraction and Immunoblotting

Whole cell lysates were obtained from cells identified in Example 6 grown in 6-well multiwell-plates using 200 μl of a RIPA Lysis and Extraction Buffer (Pierce, Rockford, Ill., USA) containing Complete Mini Protease Inhibitor cocktail (Roche Diagnostics GmbH, Germany). After removal of insoluble fragments by centrifugation, protein-concentration was quantified using the Micro BCA Protein Assay Kit (Pierce Inc., USA) according to the manufacturer's protocol.

Ten μg of whole cell lysates were mixed with lithium dodecyl sulfate (LDS) containing sample buffer and 50 mM DTT. The samples were boiled for 10 min and afterwards subjected to polyacrylamide gel electrophoresis under reducing conditions on 10% (w/w) NuPAGE Bis-TRIS gels (Invitrogen Inc., USA). Blotting was performed by the semi-dry procedure onto a nitrocellulose membrane. Antisera were diluted in TBS buffer (TRIS-Buffered Saline) containing 5% (v/v) non-fat dry milk. The following washing steps were performed in TBS puffer with 0.1% Tween® 20 (v/v). Horseradish peroxidase conjugated sheep anti-rabbit antisera (diluted 1:5000, Roche Diagnostics GmbH, Germany) was used to detect the respective primary antisera with enhanced chemiluminescence substrate (LUMI-Light plus Western Blotting substrate, Roche Diagnostics GmbH, Germany). The following primary antibody was used: rabbit polyclonal antibody against human IGF-I receptor β-chain (diluted 1:1000, sc-713; Santa Cruz Biotechnology Inc., USA).

### FACS Analysis

Surface expression of chimeric receptor was detected by flow cytometry. 1×10⁶ cells were incubated with rhuMab 2C4 (Omnitarg®, F. Hoffmann-La Roche A G, Basle Switzerland) or human IgG (1-4506, Sigma-Aldrich GmbH, Germany) as an isotype-control. Cells were stained with Phycoerythrin-conjugated goat anti-human F(ab′)₂ (Caltag Laboratories, Invitrogen Inc., USA) and expression was determined using a FACScan (Becton Dickinson, Mountain View, Calif., USA).

### Example 8

### Recombinant Production of Immunoglobulin-Conjugates Using CHO-K1 ErbB2V>E/IGF-I Cells

Plasmid p4928

For expression and production of an anti-CCR5 antibody-conjugate, light and heavy chain expression cassettes analogously e.g. as those reported in WO 2008/019817 were placed on a single expression vector in clockwise orientation. The expression vector included a neomycin resistance gene for selection.

The expression vector comprises beside the heavy and light chain expression cassette the following elements:
a nucleic acid comprising the neomycin selectable marker (neo),
an pUC-origin of replication for replication and growth of the plasmid in E. coli,
a beta-lactamase gene.

A recombinant immunoglobulin-conjugate was produced by stable transfection of CHO-K1 ErbB2V>E/IGF-I cells cultivated in ProCHO4-complete medium. Plasmid p4928 was linearized prior to transfection using the single PvuI-restriction site within the ampicillin selectable marker (gene). CHO-K1 ErbB2V>E/IGF-I cells were electroporated with the linearized DNA using the Gene Pulser XCell™ (Bio-Rad Laboratories) electroporation device with 2 mm gap electroporation cuvettes. The electroporation pulse used was 160 V/15 ms applied on 20 μg of plasmid DNA and 7.5×10⁶ cells in a total volume of 200 μl of Dulbecco's PBS. Two transfection approaches were pooled, resuspended in 37.5 ml ProCHO4-complete medium with a density of 4×10⁵ cells/ml and transferred to a CellStar T75 Flask. After 24 hours 700 μg/ml G418 sulfate (Calbiochem, La Jolla, Calif., USA) was added to the medium. The cells were transferred after 4 days into shaker flask format and cultivated for 6 generations in ProCHO4-complete selection medium with 700 μg/ml G418 sulfate prior to a single cell deposition step by flow cytometry. For this selection the FACSAria cell sorter was used to deposit a single cell into each well of twenty 96-well multiwell plates containing 50 μl cell-conditioned ProCHO4-complete medium and 50 μl fresh ProCHO4-complete medium. Two days after the single cell deposition step, 100 μl of fresh ProCHO4-complete selection medium, double concentrated with 1400 μg/ml G418 sulfate, was added. After 14 days of cultivation the IgG1 antibody concentration was analyzed in the culture supernatants by HTS-Screening with a one-step anti-human IgG ELISA. For selection of high-yield antibody production cell lines the IgG1 antibody concentration was tested again after expansion in 24-well format using a one-step anti-human IgG ELISA.

The best clone selection campaign was leading to the cell clone CHO-K1 ErbB2V>E/IGF-I 4928-3H6 with a productivity of >100 μg/ml analyzed by an analytic protein A HPLC. The cell culture conditions were as followed: shaking batch culture with a total volume of 30 ml ProCHO4-complete medium without selection agent and an initial seeding cell density of 3×10⁵ cells/ml. The immunoglobulin-conjugate containing cell culture supernatants were harvested at day 10 and were stored for 24 h at 4° C. until quantification. The integrity and molecular mass of the light and heavy chain was verified by SDS-PAGE under reduced conditions.

General information regarding the recombinant expression of human immunoglobulins is given e.g. in Meissner, P., et al., Biotechnol. Bioeng. 75 (2001) 197-203.

Quantitation of the Expressed Heavy Chain Containing Polypeptides with Anti-Human IgG ELISA

The immunoglobulin concentration in cell culture supernatants was determined by a one-step sandwich ELISA which used a biotinylated anti-human IgG F(ab′)₂ fragment as the capture reagent and for detection a peroxidase-conjugated anti-human IgG F(ab′)₂ antibody fragment.

Streptavidin coated 96-well plates (Roche Diagnostics GmbH, Germany) were coated with 2 μg/ml biotinylated goat polyclonal anti-human IgG F(ab′)₂ antibody fragment ((F(ab′)₂<h-Fcγ>Bi; Dianova, Germany, Code No. 109-066-098) capture antibody (0.1 ml/well) in diluent buffer (diluent buffer: PBS buffer containing 0.5% (w/v) bovine serum albumin) by incubation for one hour at room temperature (RT) under shaking. Thereafter, the plates were washed three times with more than 0.3 ml wash buffer (wash buffer: PBS containing 1% (w/v) Tween 20). IgG immunoglobulin-conjugate containing cell culture supernatants (samples) were diluted serially (twofold) up to a concentration of 2-10 ng/ml in diluent buffer, added to plates and incubated for one hour at RT with shaking. A purified monoclonal standard antibody (0-40 ng/ml) in diluent buffer was used for the generation of an IgG protein standard curve. After washing the plates three times with 0.3 ml/well wash buffer, bound complexes to human Fcgamma were detected with a peroxidase-conjugated F(ab′)₂ fragment of goat polyclonal anti-human F(ab′)₂-specific IgG (F(ab′)₂<h-Fcγ>POD; Dianova, Code No. 109-036-098). After washing the plates three times with 0.3 ml/well wash buffer the plates were developed with ABTS® (2,2′-azino-bis(3-ethylbenzthiazoline-6-sulfonic acid) peroxidase substrate solution (Roche Molecular Biochemicals, Code No. 1684302, Roche Diagnostics GmbH, Germany). After 10 minutes the absorbance was measured at 405 nm and 490 nm against a reagent blank (incubation buffer+ABTS solution) on a Tecan Spectrafluorplus plate reader (Tecan Deutschland GmbH, Germany). For background correction the absorbance at 490 nm was subtracted from the absorbance at 405 nm according to formula I. All samples were assayed at least as duplicates, and the values from double or triple absorbance measurements were averaged. The IgG content of the samples were calculated from a standard curve.

ΔA=(Aₛₐₘₚₗₑ⁴⁰⁵−Aₛₐₘₚₗₑ⁴⁹⁰)−(A_{blank}⁴⁰⁵−A_{blank}⁴⁹⁰) Formula I

### Quantification of Immunoglobulin Polypeptides by Affinity Binding to Protein A Sepharose

2 ml clarified culture supernatant was diluted 1:3 with ProCHO4-complete medium. Quantification of protein concentration was carried out using analytical protein-A chromatography with the Äkta Explorer 900 chromatographic system from GE Healthcare. A column packed with 250 μl protein A Sepharose™ CL-4B (GE Healthcare, Munich, Germany) was equilibrated with 2×PBS, washed with 2×PBS and 100 mM phosphate buffer (pH 5.0) and eluted with 100 mM phosphate buffer (pH 2.7). A flow rate of 0.5 ml/min and UV detection at 280 nm was employed. A purified monoclonal standard antibody diluted in ProCHO4-complete medium was used for the generation of a protein standard curve.

### SDS PAGE/Coomassie Blue Staining

The expressed and secreted polypeptides were separated by sodium dodecyl sulfate (SDS) polyacrylamide gel electrophoresis (SDS-PAGE) and stained with Coomassie blue reagent.

The culture broth containing the secreted polypeptide was centrifuged to remove cells and cell debris. An aliquot of the clarified supernatant was admixed with ¼ volumes (v/v) of NuPAGE®-LDS-Sample-Puffer (Invitrogen Corp., USA) and 1/10 volume (v/v) of NuPAGE®-10× Reducing Agent (Invitrogen Corp., USA). The samples were incubated for 10 min. at 70° C. and afterwards the protein was separated by SDS-PAGE. The NuPAGE® Pre-Cast gel system was used according to the manufacturer's instruction. In particular, 10% NuPAGE® Novex® Bis-TRIS Pre-Cast gels (pH 6.4) and a NuPAGE® MOPS running buffer was used in combination with a NuPAGE® Antioxidant.

After separation for 1 h at 120V the gel was stained in Coomassie blue solution (30% (v/v) methanol; 10% (v/v) acetic acid; 0.2% (w/v) Coomassie Brilliant Blue R-250 Dye, Pierce, Rockford, Ill., USA) for 1 h with gentle agitation and destained overnight in water.

### Example 9

### Cell Density in Cultivation

The cell clones CHO-K1-5519-1B6-18B3 and CHO-K1-5519-1B6-17C5 obtained in Example 4 were cultivated in ProCHO4 medium supplemented with 1×HT supplement, 6 mM glutamine, and 4 μg/ml puromycin in 125 shaker flasks with 160 rpm. As reference CHO-K1 adapted to serum free suspension culture was also cultivated under the same conditions except that no puromycin was used but 50 ng/ml IGF-1 were added. The cultures were inoculated with approximately 2 to 3×10⁵ cells/ml in a total volume of 62.5 ml.

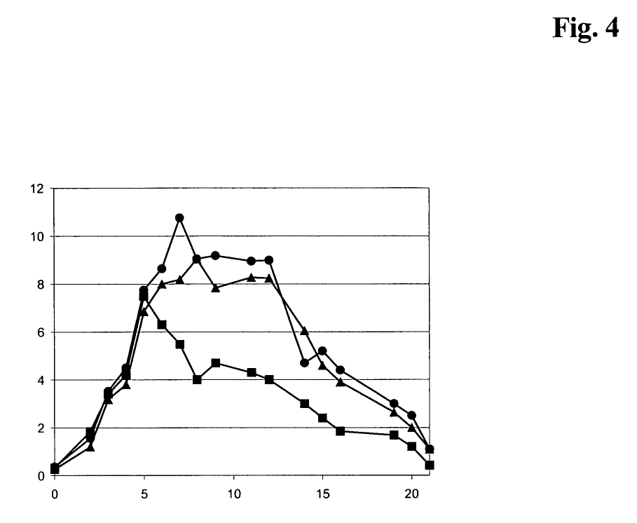

For cell density analysis a sample of 0.5 to 1.0 ml was taken every day from the cultures. Cell clumps were dissolved by incubating 200 μl of the cell suspension with 20 μl Trypsin for 30 min. at 37° C. For measurement 50 or 100 μl of the disaggregated cell suspension was diluted in 10 ml CASYton-buffer (Schärfe Systems GmbH, Germany) and the total viable cell-concentration in 400 μl thereof was determined in triplicate by electronic pulse area analysis using the Cell Counter and Analyzer-System CASY (Schärfe System GmbH, Germany). The results are shown in Table 2 and FIG. 4.

TABLE 2
Cell density in 10⁶ cells/ml.

| | | CHO-K1-5519- | CHO-K1-5519- |
|---|---|---|---|
| | CHO-K1 | 1B6-18B3 | 1B6-17C5 |
| day | [×10⁶ cells/ml] | [×10⁶ cells/ml] | [×10⁶ cells/ml] |
| 0 | 0.315 | 0.365 | 0.258 |
| 2 | 1.840 | 1.560 | 1.200 |
| 3 | 3.400 | 3.520 | 3.180 |
| 4 | 4.200 | 4.500 | 3.800 |
| 5 | 7.480 | 7.750 | 6.850 |
| 6 | 6.300 | 8.640 | 8.000 |
| 7 | 5.480 | 10.760 | 8.190 |
| 8 | 4.000 | 9.050 | 9.040 |
| 9 | 4.700 | 9.180 | 7.840 |
| 11 | 4.300 | 8.950 | 8.280 |
| 12 | 4.000 | 8.990 | 8.240 |
| 13 | 2.400 | 3.700 | 3.300 |
| 14 | 3.000 | 4.700 | 6.050 |
| 15 | 2.400 | 5.200 | 4.600 |
| 16 | 1.840 | 4.400 | 3.900 |
| 19 | 1.680 | 3.000 | 2.640 |
| 20 | 1.200 | 2.500 | 2.000 |
| 21 | 0.420 | 1.100 | 1.100 |

1. A CHO cell adapted to growth in serum free medium, wherein said CHO cell expresses a chimeric ErbB2^{V→E}/IGF-I receptor.

2. The CHO cell of claim 1, wherein said CHO cell is a CHO-K1 cell.

3. The CHO cell of claim 1, wherein said cell comprises a constitutively active mitogenic receptor, which is a fusion polypeptide comprising amino acids 1-682 of SEQ ID NO: 09 with the mutation V659E and amino acids 929-1337 of SEQ ID NO: 10.

4. The CHO cell of claim 1, wherein said cell comprises a nucleic acid encoding the amino acid sequence of SEQ ID NO: 03.

5. The CHO cell of claim 1, wherein said cell is growing in suspension.

6. The CHO cell of claim 1, wherein said CHO cell grows in cultivation to a maximum cell density of at least 8×10⁶ cells/ml and said maximum cell density is reached within 8 to 12 generations starting from a cell density of 2×10⁵ to 3×10⁵ cells/ml in a volume of 60 to 70 ml.

7. The CHO cell of claim 6, wherein said cultivation of said CHO cell is a fed-batch cultivation.

8. The cell line DSM ACC2851.

9. A method for obtaining a CHO cell with a constitutively active mitogenic receptor, wherein said method comprises
a) transfecting a CHO cell with a first nucleic acid comprising
i) an expression cassette encoding a selectable marker flanked by two loxP-sites,
ii) an expression cassette for the expression of a constitutively active mitogenic receptor of the amino acid sequence of SEQ ID NO: 03,
b) selecting a CHO cell transfected with said first nucleic acid for the absence of the selectable marker comprised in the first nucleic acid and that has a constitutively active mitogenic receptor of the amino acid sequence of SEQ ID NO: 03,
c) transfecting the CHO cell selected in step b) with a second nucleic acid comprising a CRE recombinase expression cassette; and
d) selecting a CHO cell transfected with the second nucleic acid as a CHO cell with a constitutively active mitogenic receptor.

10. The method of claim 9, wherein said first nucleic acid comprises an expression cassette for the expression of transferrin.

11. The CHO cell of claim 1, wherein said CHO cell is expressing a heterologous polypeptide.

12. A method for the production of a heterologous polypeptide, comprising:
a) providing a CHO cell according to claim 1,
b) transfecting said CHO cell with a nucleic acid comprising
i) a first expression cassette comprising a nucleic acid encoding said heterologous polypeptide,
ii) a second expression cassette encoding a selectable marker,
c) cultivating said transfected CHO cell under conditions suitable for the expression of said heterologous polypeptide; and
d) recovering said expressed heterologous polypeptide from the culture medium or the cells.

13. The method of claim 12, wherein said heterologous polypeptide is selected from the group consisting of immunoglobulins, immunoglobulin heavy chains, immunoglobulin light chains, immunoglobulin fragments, or immunoglobulin conjugates.
